(19) United States
(12) Patent Application Publication
(10) Pub. No.: US 2022/0235014 A1
(43) Pub. Date: Jul. 28, 2022

(54) PROCESS FOR THE PREPARATION OF HIGHLY PURE ERLOTINIB HYDROCHLORIDE

(71) Applicants: AKSHAY KANT CHATURVEDI, Bhiwadi (IN); SATYENDRA SINGH, Bhiwadi (IN); BIJAN PANDA, Bhiwadi (IN)

(72) Inventors: AKSHAY KANT CHATURVEDI, Bhiwadi (IN); SATYENDRA SINGH, Bhiwadi (IN); BIJAN PANDA, Bhiwadi (IN)

(73) Assignee: SHIVALIK RASAYAN LIMITED, Bhiwadi (IN)

(21) Appl. No.: 17/156,526

(22) Filed: Jan. 23, 2021

(51) Int. Cl. C07D 239/94; C07C 253/30; C07C 249/02

(52) U.S. Cl. CPC C07D 239/94; C07B 2200/13; C07C 249/02; C07C 253/30

(57) ABSTRACT

The present invention provides to an improved process for the preparation of highly pure Erlotinib hydrochloride of formula (I)

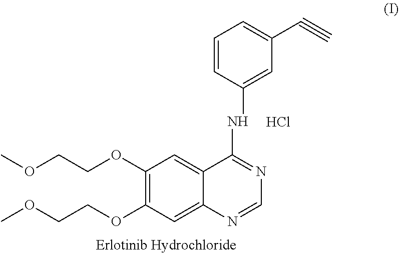 [SMILES: C#Cc1cccc(Nc2ncnc3cc(OCCOC)c(OCCOC)cc23)c1 ; Cl]

The substantially pure Erlotinib hydrochloride (I) obtained by improved process of the present invention is having purity of greater than 99.8% (% w/w by HPLC).
Erlotinib hydrochloride is useful in the treatment of cancer more particularly in the treatment of lung cancer and pancreatic cancer.

### FIELD OF THE INVENTION

The present invention relates to an improved process for the preparation of highly pure Erlotinib hydrochloride (I)

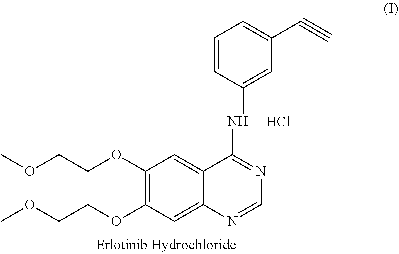 [SMILES: C#Cc1cccc(Nc2ncnc3cc(OCCOC)c(OCCOC)cc23)c1 ; Cl]

### BACKGROUND OF THE INVENTION

Erlotinib hydrochloride (I) is chemically named as N-(3-ethynylphenyl)-6, 7-bis (2-methoxy ethoxy)-4-quinazolinamine hydrochloride

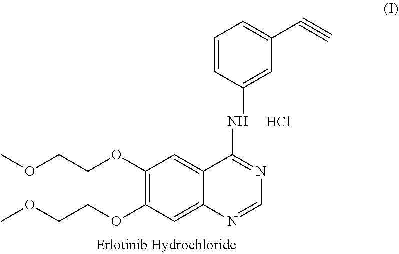 [SMILES: C#Cc1cccc(Nc2ncnc3cc(OCCOC)c(OCCOC)cc23)c1 ; Cl]

Erlotinib hydrochloride (I) is an inhibitor of oncogenic and proto-oncogenic protein tyrosine kinases viz, Epidermal growth factor receptor (EGFR).

Erlotinib is therefore useful in the treatment of proliferative disorders and is currently marketed as Xeloda® for the treatment of lung cancer and pancreatic cancer.

It is indicated for the treatment of patients with locally advanced or metastatic non-small cell lung cancer after failure of at least one prior chemotherapy regimen, and in combination with gemcitabine is indicated for the first-line treatment of patients with locally advanced, unresectable or metastatic pancreatic cancer.

Schnur, et al in U.S. Pat. No. 5,747,498B2 discloses a process for the preparation of Erlotinib free base and its HCl salt, which follows the pathway as given in the Scheme-1.

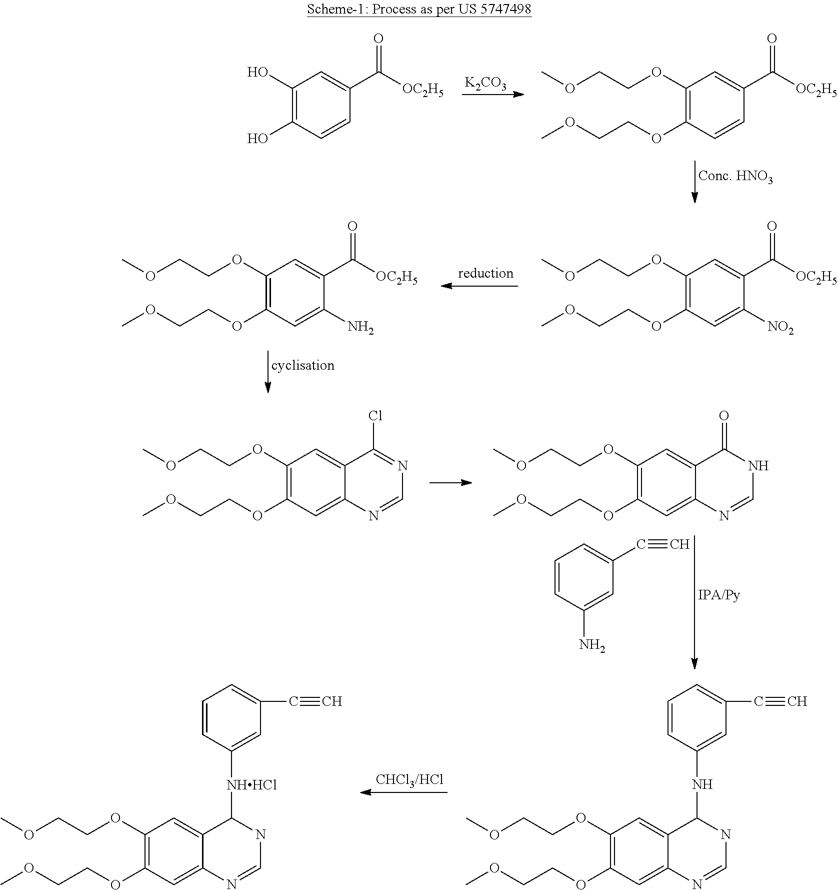

Kalvins hars et al in WO2015023170A2 discloses the preparation method of Erlotinib hydrochloride according to the pathway as given in the Scheme-2:

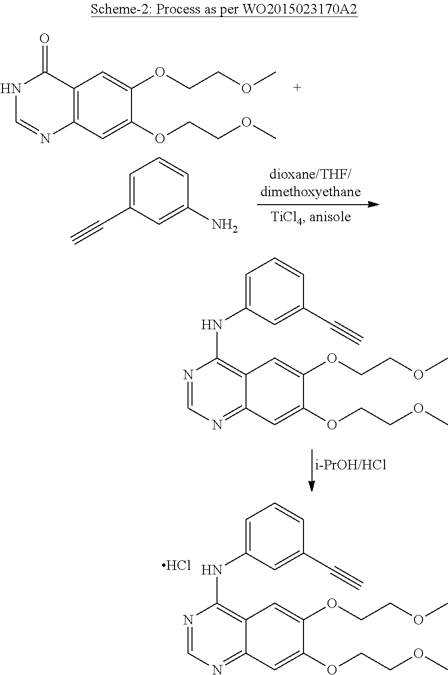

Murugesan et al in U.S. Pat. No. 8,440,823B2 disclosed the preparation method of Erlotinib hydrochloride according to the pathway as given in the Scheme-3:

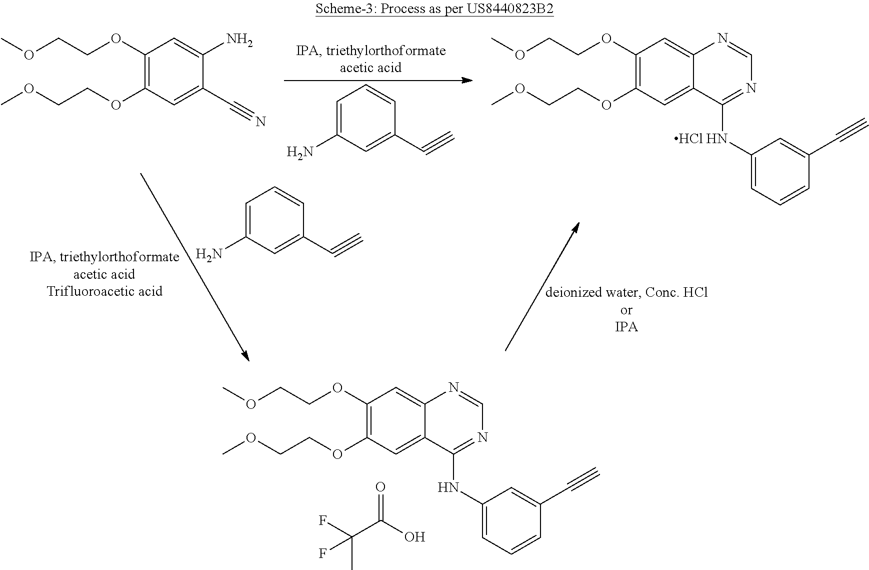

Baratella et al in U.S. Pat. No. 9,428,468B2 discloses the preparation method of Erlotinib hydrochloride according to the pathway as given in the Scheme-4:

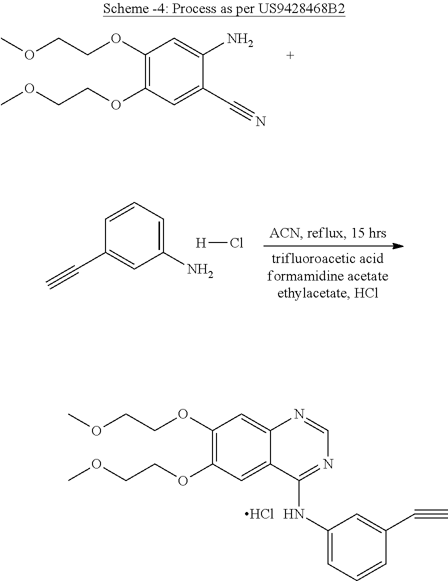

Simhadri Srinivas et al in WO2013156835A2 discloses the preparation method of Erlotinib hydrochloride according to the pathway as given in the Scheme-5:

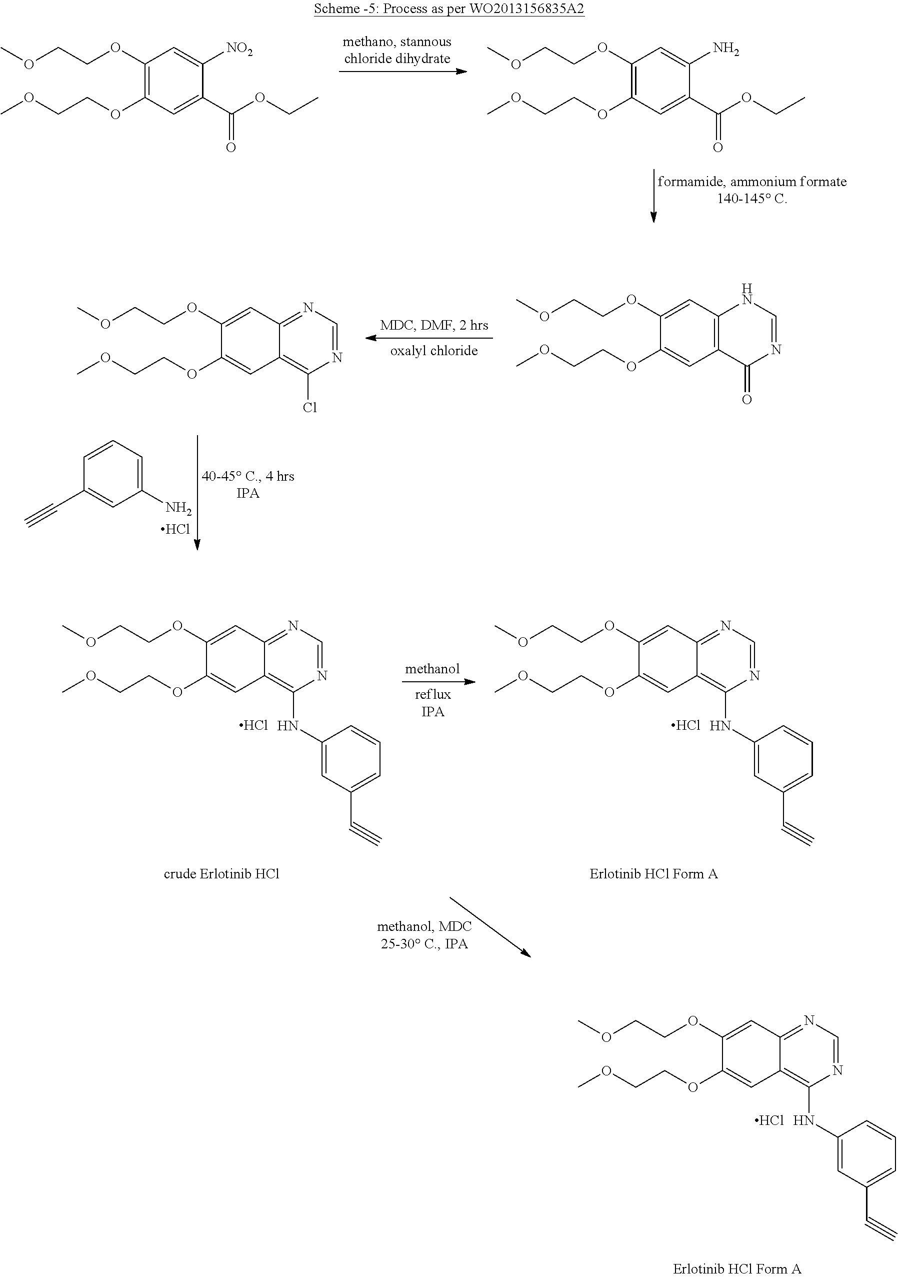

Gore et al in U.S. Pat. No. 8,952,022B2 discloses the preparation method of Erlotinib hydrochloride according to the pathway as given in the Scheme-6:

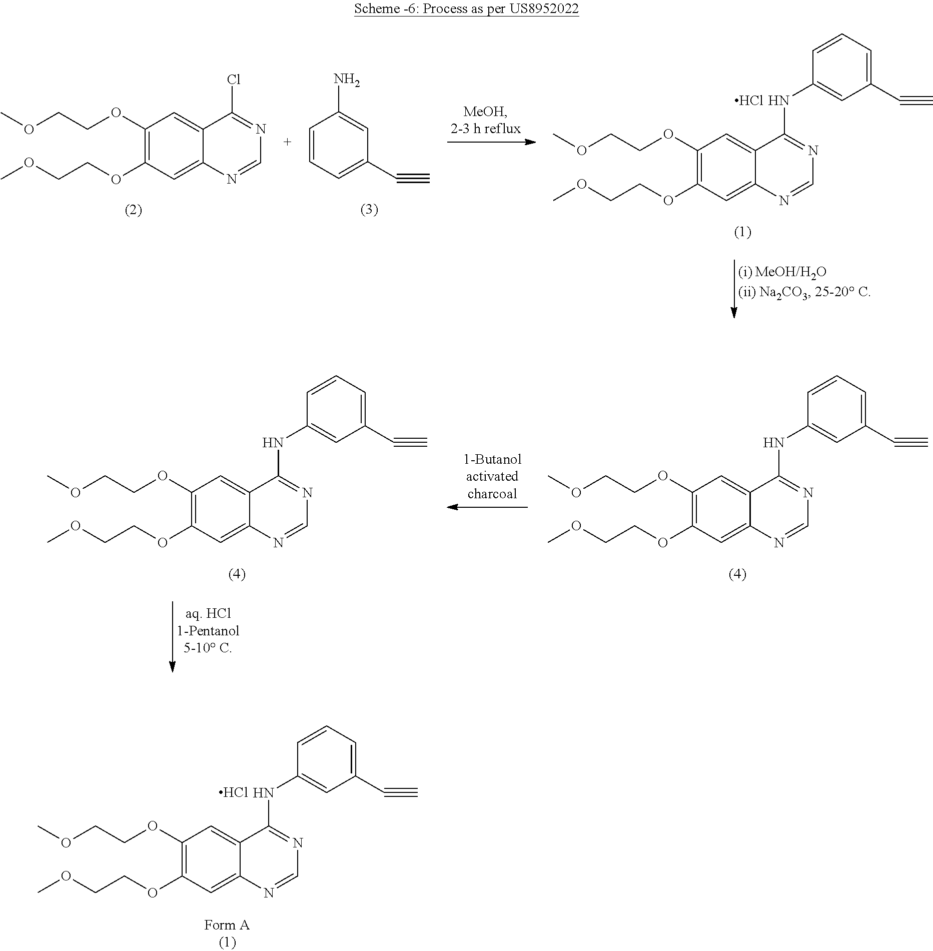

The present inventors has repeated the above process and found the following disadvantages: In most of the patent literature, the rate of reaction is very low, which parallel yield in the formation of by-products, which is tedious for the removal as well as unwanted reactions are observed during the formation of Erlotinib, as the reaction in carried out at very high temperature.

In view of the above, to overcome the prior-art problems the present inventors have now developed an improved process for the preparation of Erlotinib hydrochloride, using industrially feasible and viable process, with the use of industrially friendly solvents, which does not include tedious work up.

Being Erlotinib hydrochloride as an important anticancer therapeutic agent, additional and improved ways of preparing Erlotinib hydrochloride salt may be of immense value to pharmaceutical science and the healthcare of cancer patients. Hence, there exists a need in the development of economically viable processes providing highly pure active, which may be commercially up scalable, viable, safer for handling, less time consuming and with better and consistent quality parameters.

The present inventors have now developed an improved process for the preparation of highly pure Erlotinib hydrochloride (I), which is stable and devoid of process related impurities.

### SUMMARY OF THE INVENTION

Particular aspects of the present invention relates to a process for the preparation of Erlotinib or its hydrochloride salt of Formula (I).

Different aspects of the present application are summarized herein below individually.

In one aspect of the present application, the present invention relates to a process for preparation of highly pure or substantially Erlotinib hydrochloride (I),

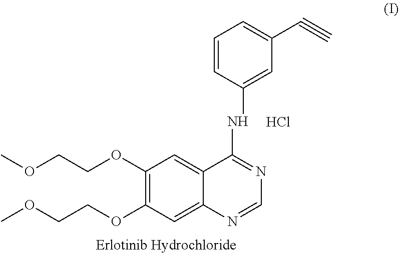 [SMILES: C#Cc1cccc(Nc2ncnc3cc(OCCOC)c(OCCOC)cc23)c1 ; Cl]

comprising the steps of
a) aminating the compound of formula (A) at temperature ranging between 45-65° C. in the presence of alkali metal dithionite and an organic solvent to obtain the compound of formula(B)

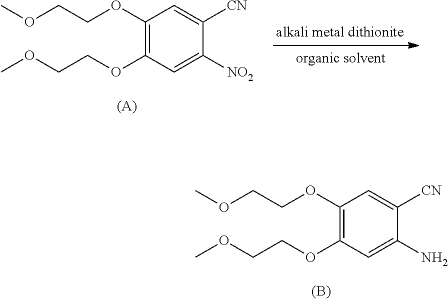

b) reacting compound of formula (B) with N, N-Dimethyl formamide dimethyl acetal (DMFDMA) in the presence of non-polar organic solvent and an organic acid to get an intermediate of compound of formula (C), which in-situ reacted with ethynylaniline hydrochloride to isolate the compound of formula (D)

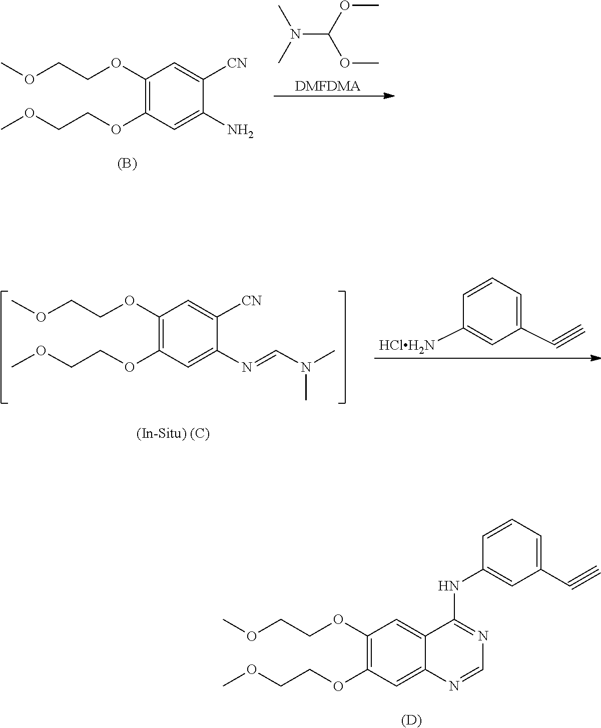

c) hydrochlorinating the compound of formula (D) in polar organic solvent to get crystalline Erlotinib hydrochloride (I).

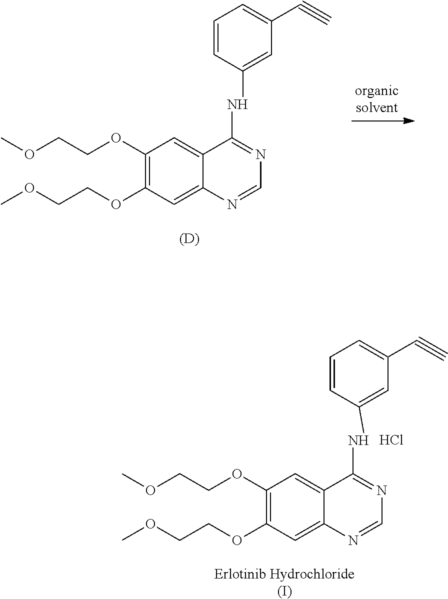

In yet another aspect according to the present invention, it relates to highly pure or substantially pure Erlotinib hydrochloride having purity of greater than 99.8% (% w/w by HPLC). This crystalline form is thermodynamically stable and designated as Form SEA.

Further specific aspects of the invention are detailed in the description part of the specification, wherever appropriate.

### BRIEF DESCRIPTION OF THE DRAWINGS

FIG. 1: is an illustration of a High performance Liquid Chromatography (“HPLC”) of Erlotinib hydrochloride Form SEA obtained in the initial stage according to the present invention.

FIG. 2: is an illustration of a High performance Liquid Chromatography (“HPLC”) of Erlotinib hydrochloride Form SEA obtained after 9 months stability (30° C./65±5% condition) according to the present invention.

FIG. 3: is an illustration of a differential scanning calorimetric (“DSC”) curve of Erlotinib hydrochloride Form SEA obtained according to the present invention.

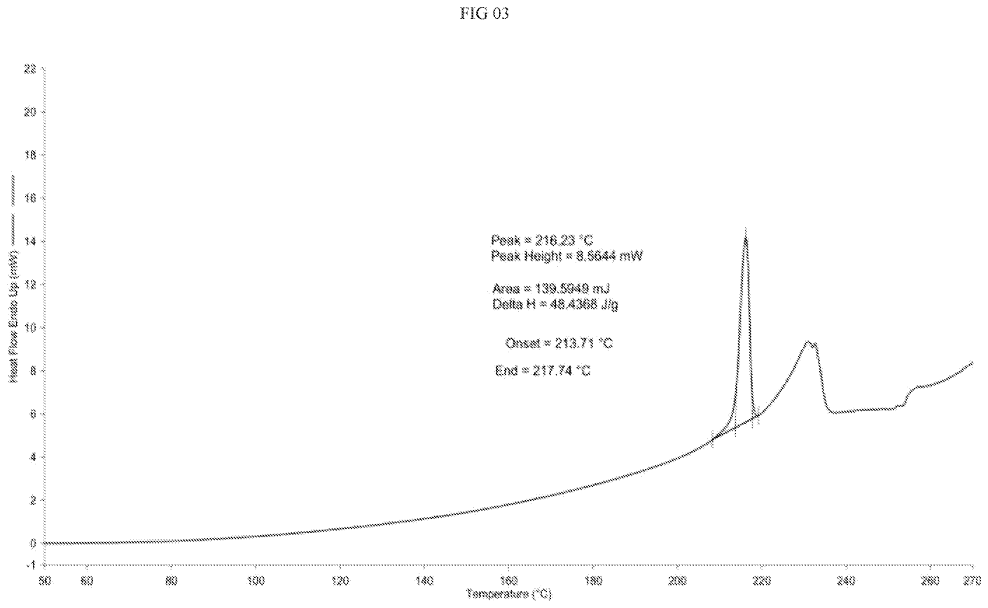

FIG. 4: is an illustration of X-ray powder diffraction (XRPD) pattern of Erlotinib hydrochloride Form SEA obtained in the initial stage according to the present invention.

FIG. 5: is an illustration of X-ray powder diffraction (XRPD) pattern of Erlotinib hydrochloride Form SEA obtained after 9 months stability (30° C./65±5% condition) according to the present invention.

### DETAILED DESCRIPTION

As set forth herein, embodiments of the present invention relate to a process for preparation of Erlotinib hydrochloride (I). The present invention deals with a simple and industrially amenable process for making the compound of formula (I), which exhibits various advantages over other processes known in the state of arts. The advantages are discussed on the relevant places of further description. Individual embodiments of the present invention are detailed herein below separately.

In one embodiment according to the present application, it provides a process for preparing highly pure Erlotinib or its hydrochloride salt of Formula (I).

In another embodiment according to present application, it provides a process for preparation of highly pure or substantially pure Erlotinib or its hydrochloride salt of Formula (I),

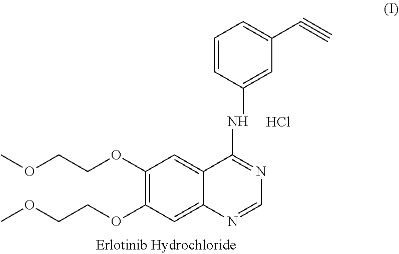 [SMILES: C#Cc1cccc(Nc2ncnc3cc(OCCOC)c(OCCOC)cc23)c1 ; Cl]

comprising the steps of:
a) aminating the compound of formula (A) at temperature ranging between 45−65° C. in the presence of alkali metal dithionite and an organic solvent to obtain the compound of formula (B)

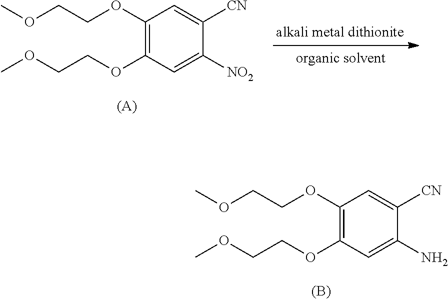

b) reacting compound of formula (B) with N, N-Dimethyl formamide dimethyl acetal (DMFDMA) in the presence of non-polar organic solvent and an organic acid to get an intermediate of compound of formula (C), which in-situ reacted with ethynylaniline hydrochloride to isolate the compound of formula (D)

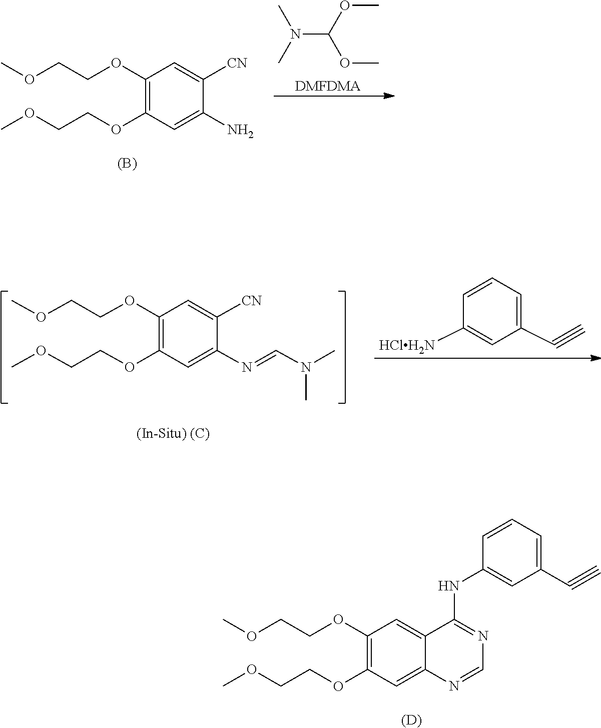

c) hydrochlorinating the compound of formula (D) in polar organic solvent to get Erlotinib hydrochloride (I).

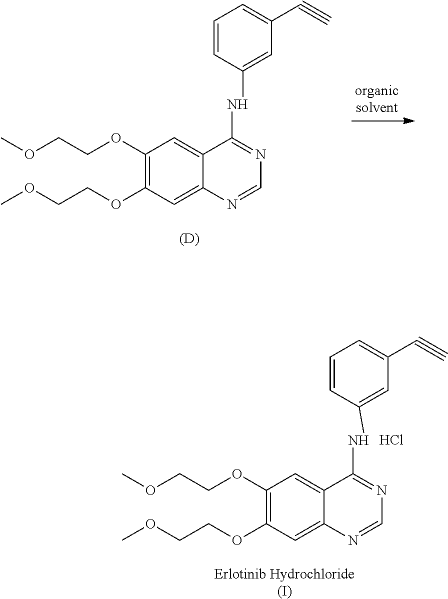

Individual steps of the embodiments are detailed herein below.

In step a) process of aminating the 4, 5-bis (2-methoxyethoxy)-2-nitrobenzonitrile (A) is carried out at temperature ranging between 45-65° C. in the presence of alkali metal dithionite and an organic solvent to obtain 4, 5-bis (2-methoxyethoxy)-2-aminobenzonitrile (B). The alkali metal dithionite used is selected from sodium or potassium dithionate.

The addition of the alkali metal dithionite is preferably carried out in two or more different lots in order to ensure improved yields, since single lot dumping of alkali metal dithionite resulted in significantly reduction of yield (yield >50% loss) and process efficiencies including high exothermicity.

The organic solvent used is selected from ethylacetate, dichloromethane, chloroform, carbon tetrachloride or a combination thereof. The organic solvent used in this step is for the for the purpose of extraction of aminated compound of Formula (B).

In step b), reaction is carried out of 4, 5-bis (2-methoxyethoxy)-2-aminobenzonitrile (B) with N, N-Dimethyl formamide dimethyl acetal (DMFDMA) in the presence of non-polar organic solvent and an organic acid to get an intermediate of compound of formula (C), which is further in-situ reacted with ethynylaniline hydrochloride to isolate the compound of formula (D). The organic acid used is selected from aliphatic acid like acetic acid, propionic acid, butanoic acid, citric acid or tartaric acid.

In situ reaction has advantages over handling multiple steps of isolation and subsequently processing for the next stages besides improved yields/efficiencies and minimizing steps of plant operations. The present invention resides in avoiding step of isolating an intermediate (C) without compromising on the quality characteristics of final API (Active Pharmaceutical Ingredient) and improved process efficiencies and yields.

The non-polar organic solvent is selected from toluene (methyl benzene), ethyl benzene or xylene.

The step b) is advantageously carried out at temperature ranging between 95° C. to 120° C., while necessary to obtain the desired quality and yields. It was surprisingly observed by inventors that reaction carried out lower temperatures does not results in acceptable quality of intermediate which was found to generate impurities in the subsequent stages more particularly process related impurities. Hence a control on process parameters was observed significantly important by inventors of the present application.

In the subsequent step of the process of the present invention a treatment with a source of hydrochloric acid is carried out to obtain Erlotinib hydrochloride.

Step c) provides process for the preparation of Erlotinib hydrochloride from Erlotinib free base (D) which involved use of a polar organic solvent selected from isopropanol, ethanol, dimethylformamide or methyl isobutyl ketone (MIBK) or mixture thereof.

In a particular embodiment, the solvent used was isopropanol or a mixture of IPA and MIBK and 10-15% IPA-HCl solution was added slowly i.e. not less than 2-5 hours at a temperature ranging between 60° C. to 65° C.

In step c), the hydrochloride salt formed—the washing of the cake (wet API) was conducted with a mixture of isopropyl alcohol and methyl isobutyl ketone (MIBK) which may be in the ratio ranging between 20-70:80-30 respectively.

In a particular embodiment, the ratio of solvent used for washing was about 50:50 v/v.

The product obtained by the present invention is free of process related impurities, including unreacted intermediates, side products, degradation products and other medium dependent impurities. The Erlotinib and its hydrochloride salt obtained by the process according to the present invention is highly pure having a substantially purity of greater than 99.8% and having total impurities of ˜0.2% selected from A and/or C and process related reactants (key starting material) and intermediates.

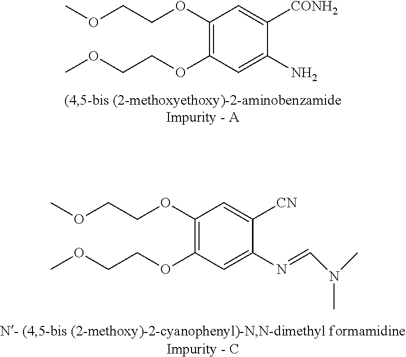

In yet further embodiment the steps of combining isopropanol and Hydrochloride mixture, it comprises of slow addition of isopropanol (IPA) and hydrochloride mixture, wherein isopropanol and hydrochloride prepared earlier by combining Hydrochloride gas and isopropanol comprises of hydrochloride strength ranging between 5 to 20% w/v.

After combining this acidic alcohol mixture, the solution may preferably be maintained under stirring for a time duration between 10-45 minutes for complete hydrochlorination of Erlotinib base and in order to get the higher yield of Erlotinib hydrochloride salt.

The step of cooling the reaction mixture may be carried out for the mixture upto about 25-30° C. to attain the crystalline material precipitated out with no or minimal possible degradation for achieving the pure crystal form.

In another embodiment of the present invention, it provides a highly pure or substantially pure Erlotinib hydrochloride having purity exceeding of 99.8% (% w/w by HPLC).

The obtained crystalline form is a thermodynamically stable Form SEA resembling with the innovator's Form A which is disclosed in U.S. Pat. No. 6,900,221, except few minor peaks and the lack of stability in Form A.

Form SEA exhibits an X-ray powder diffraction pattern having characteristic diffraction angle peaks expressed in 20° at approximately 5.72, 9.89, 11.39, 18.95, 22.84, 23.60, 24.28, 25.47 and 29.31±0.2 0° and having the melting point ranging between 216-218° C.

Form SEA is an anhydrous form and exists in the substantially pure form with purity exceeding 99.8% (% w/w by HPLC)

In order to check the thermodynamic stability inventors of the present application performed 9 months stability in the following storage conditions:

| S. No. | Temperature | Relative Humidity |
|---|---|---|
| 1. | 30° C. ± 2° C. | 65% ± 5% |
| 2. | 40° C. ± 2° C. | 75% ± 5% |

The below mentioned table shows the thermodynamic stability nature of the polymorphic Form SEA and the HPLC purity indicates to retain substantially pure form nature with purity exceeding 99.8% (% w/w by HPLC).

| | Limits | Initial | 1M | 2M | 3M | 6M | 9M |
|---|---|---|---|---|---|---|---|
| Moisture | Not more | 0.17% | 0.24% | 0.26% | 0.21% | 0.17% | 0.42% |
| Content | than | w/w | w/w | w/w | w/w | w/w | w/w |
| (by KF | 0.5% | | | | | | |
| method) | w/w | | | | | | |
| Assay (by | >99.8% | 100.84% | 99.19% | 101.56% | 101.41% | 99.99% | 100.40% |
| HPLC; on | | w/w | w/w | w/w | w/w | w/w | w/w |
| anhydrous | | | | | | | |
| basis) | | | | | | | |

| | | |
|---|---|---|
| XRPD | Initial peaks: | After nine months at |
| peaks | 5.72, 9.89, 11.39, 18.95, 22.84, | 30° C./65 ± 5% condition |
| | 23.60, 24.28, 25.47 and | 5.62, 9.78, 11.30, 18.85, 22.74, |
| | 29.31 ± 0.2 θ° | 23.49, 24.19, 25.34 and |
| | | 29.189 ± 0.2 θ° |
| XRPD | Initial peaks: | After nine months at |
| peaks | 5.72, 9.89, 11.39, 18.95, 22.84, | 40° C./75 ± 5% condition |
| | 23.60, 24.28, 25.47 and | 5.65, 9.82, 11.32, 18.92, 22.77, |
| | 29.31 ± 0.2 θ° | 23.54, 24.21, 25.39 and |
| | | 29.27 ± 0.2 θ° |

The crystalline polymorphic form A as reported in U.S. Pat. No. 6,900,221B1 indicates XRPD 2° θ additional refection peaks at 6.2, 7.7, 8.0 and 8.7. However said diffraction angle peaks were found absent in the thermodynamically stable crystalline form designated form as Form SEA which was stable up to 12 months under storage conditions of 30° C./65±5% and 40° C./75±5%. This indicates that the crystalline form obtained according to the present invention as Form SEA is different from reported Form A as per U.S. Pat. No. 6,900,221B1.

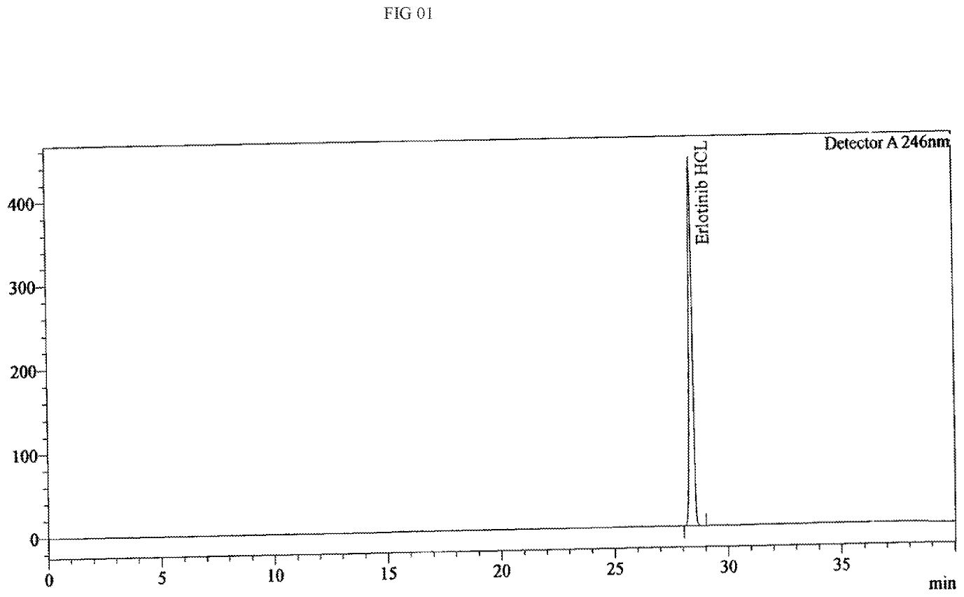

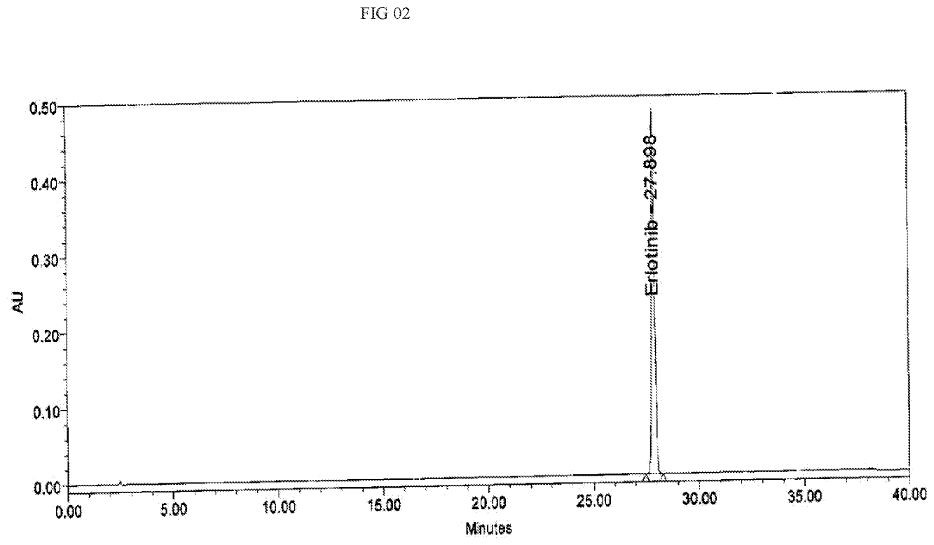

FIG. 1 and FIG. 2 illustrate the High performance Liquid Chromatography (“HPLC”) of Erlotinib hydrochloride Form SEA obtained in the initial stage and after 9 months stability respectively. Figures reveal that the purity remains same and the polymorphic form Form SEA is a substantially pure form with purity exceeding 99.8% (% w/w by HPLC).

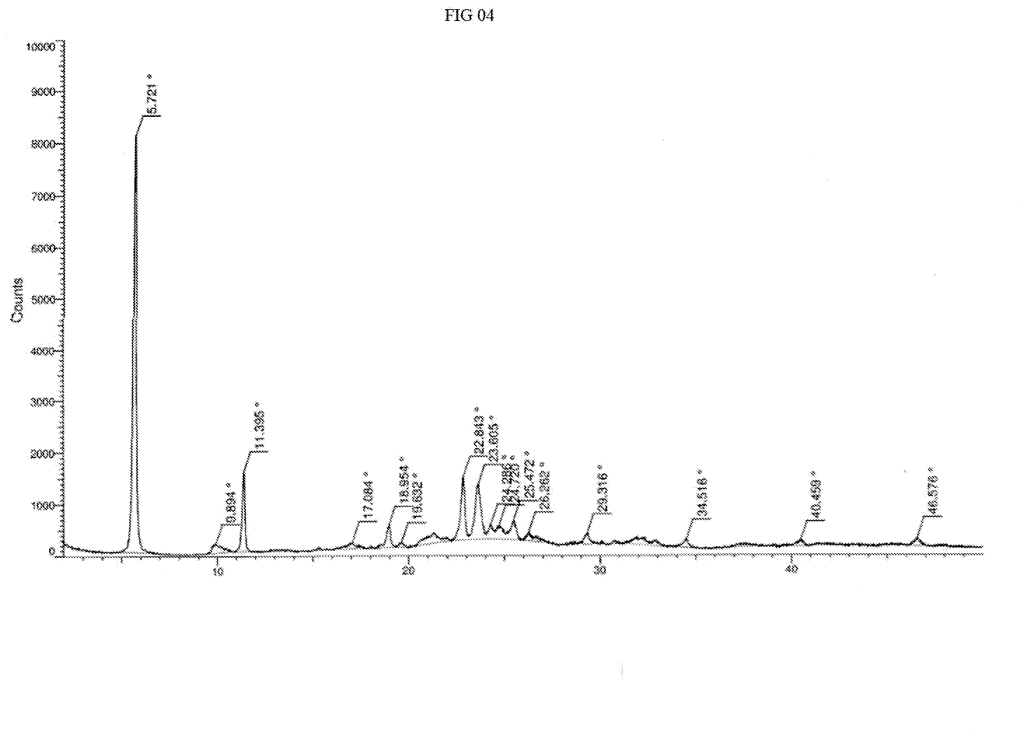

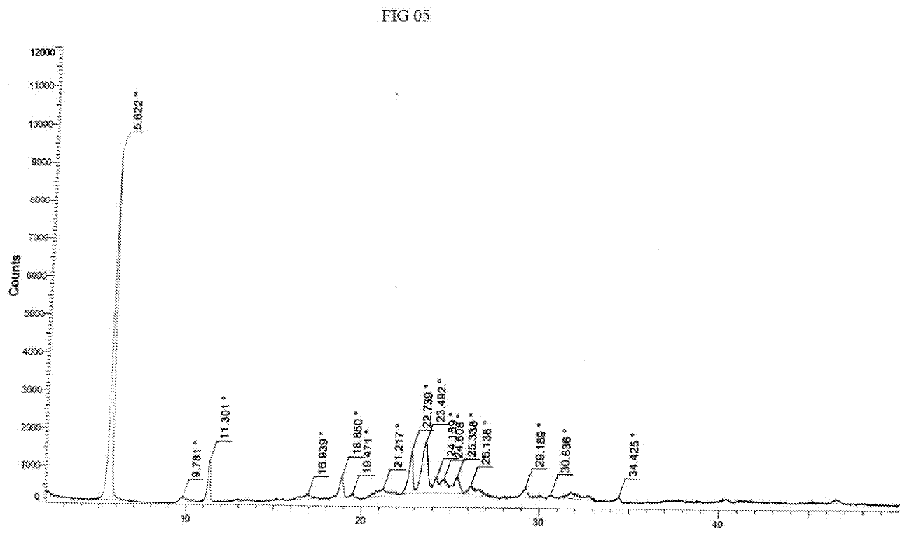

FIG. 4 and FIG. 5 illustrate the of X-ray powder diffraction (XRPD) pattern of Erlotinib hydrochloride Form SEA obtained in the initial stage and after 9 months stability respectively. Figures reveal that the crystalline nature of the material remains same and thermodynamic stable.

The process related impurities, including unreacted intermediates, side products, degradation products and other medium dependent impurities, that appears in the impurity profile of the Erlotinib hydrochloride can substantially be removed by the process of the present invention resulting in the formation highly pure Erlotinib, which parallel leads to the formation of highly pure and stable Erlotinib hydrochloride.

Erlotinib hydrochloride obtained according to present invention shall be dried under vacuum to attain water content in the range between 0.1 to 0.5% w/w.

In yet further another embodiment isolation of the Erlotinib and Erlotinib hydrochloride may be carried out by filtration, solvent removal (extraction), layer separation, concentration, distillation, or a combination thereof.

Process of the present invention avoids the formation of by-products and process related impurities in the formation of substantially pure Erlotinib or hydrochloride salt.

In yet further another embodiment, it provides that the Erlotinib hydrochloride obtained by the processes of the present application may be formulated as solid compositions for oral administration in the form of capsules, tablets, pills, powders or granules useful in the treatment lung cancer and pancreatic cancer.

Erlotinib hydrochloride of the present invention may have one or more advantageous and desirable properties compared to the known Erlotinib hydrochloride, which are not limited to better stability, solubility and quality parameter leading to improved storage and distribution.

In another embodiment, the Erlotinib hydrochloride obtained by the processes of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Erlotinib Hydrochloride of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions derived from Erlotinib hydrochloride of the present application may also comprise to include the pharmaceutically acceptable carrier used for the preparation of solid dispersion, wherever utilized in the desired dosage form preparation.

While the present invention has been described in terms of its specific embodiments, certain modifications and equivalents will be apparent to those skilled in the art and are intended to be included within the scope of the present invention.

Certain specific aspects and embodiments of the present application will be explained in more detail with reference to the below examples, which are provided by way for illustration purpose only and should not be construed as limiting the scope of the invention in any manner.

The following examples illustrate the nature of the invention and are provided for illustrative purposes only and should not be construed to limit the scope of the present invention.

### Example

### Step-a) Preparation of 4, 5-bis (2-methoxyethoxy)-2-aminobenzonitrile (B)

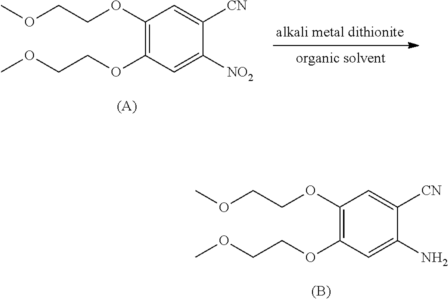

Charged first lot of 4,5-bis-(2-methoxyethoxy)-2-nitrobenzo nitrile (100 gm) of the compound of formula (A), Purified water (1682 ml) and sodium dithionite (187 gm) at 20-30° C. in a reaction flask. Stirred the reaction mass for 5-10 min. Charged second lot (48 gm) of 4, 5-bis-(2-methoxyethoxy)-2-nitrobenzo nitrile of the compound of formula (A) at 20-30° C. Added purified water (18 ml) at 20-30° C. The reaction mixture was heated at 50-55° C. and maintained this temperature for 1.0 hr. Raised the temperature of reaction mass to 60-65° C. Added conc hydrochloric acid (208.4 ml) in 45-60 min at 60-65° C. Stirred the reaction mass for 30 min. at 60-65° C. The reaction mass was cooled down to 15-20° C. and used sodium hydroxide solution (215 ml) to adjust the pH of the reaction mass to 10-12. Raised the temperature of reaction mass to 25-30° C. (if not in range). Charged ethyl acetate (804 ml) and stirred for 30 min at 25-30° C. and separate the organic layer. The aqueous layer was extracted two times with ethyl acetate (350 ml). Combined all three ethyl acetate layers and charged purified water (252 ml) for washing. Stirred for 30 min at 30° C. and separate the organic layer. Washed the organic layer with brine solution (504 ml). Stirred the mass for 30 min at 30° C. Add activated Charcoal (10 g) and stirred for 30 min at 25-30° C. Filtered the mass through hyflo and washed by ethyl acaetate (196 ml). Distilled out ethyl acetate under vacuum below 55° C. Cooled the residue to 25-30° C. Charged hexane (400 ml) and stirred for 1 hr at 30° C. Filtered and washed by 47 ml ethyl acetate. Dried the material for 12 hrs at 50-55° C.

Yield: Dry wt 85 gm (95%)

### Step-b) Preparation of Erlotinib (D)

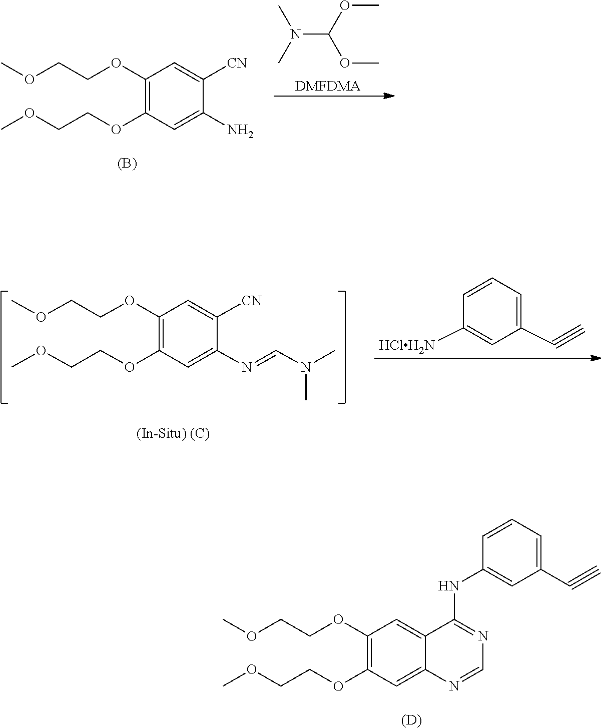

Charged 4, 5-bis (2-methoxyethoxy)-2-aminobenzonitrile (B) (100 g), toluene (1000 ml) and N, N-Dimethyl formamide dimethyl acetal (DMFDMA) (100 ml) into a reaction flask under stirring at 20-30° C. Added 1.4 ml Acetic acid at 20-30° C. Raised the temperature of reaction mass to 105-110° C. and maintained for 2 hr.

Cool the reaction mass to 95-100° C. Distilled out the toluene completely under vacuum at temperature below 100° C. Degassed the material for 30 min at 95-100° C. Cool the reaction mass to 25-30° C.

Charged Acetic acid (741.7 ml) and Ethynyl aniline hydrochloride (57.78 g) under stirring into the reaction flask in 30 min. at 20-30° C. Raised the temperature of reaction mass to 115-120° C. and maintained it for 4 hr. Cooled down the reaction mass to 90-95° C. and distilled toluene completely under vacuum at temperature below 95° C. The reaction mass cooled to 25-30° C. and added purified water (1900 ml). Stirred the reaction mass for 30 min at 25-35° C. Cooled the reaction mass to 15-20° C. and pH was adjusted to 9.0-10.0 using 20-25% of 300 ml Aqueous ammonia at 15-20° C. Raised the temperature to 25-35° C. and added ethyl acetate (500 ml) at the same temperature. Stirred the reaction mass for 2.0 hours at 25-35° C. Filtered and washed the material with purified water (670 ml) at 25-35° C. Suck dried for 1-2 hours and unload the wet material.

Charged purified water (1500 ml) in another reactor and added the wet material at 25-30° C. Stirred the reaction mass at 25-35° C. Charged aqueous ammonia solution (32 ml) at 25-35° C. Stir the reaction mass for 2.0 hours at 25-35° C. Filtered and washed the material with purified water (670 ml) at 25-35° C. Suck dried for 1-2 hours and unload the wet material.

Charged purified water (1500 ml) in another reactor. Charged the wet material at 25-30° C. Stirred the reaction mass at 25-35° C. Charged aqueous ammonia solution (32 ml) at 25-35° C. Stirred the reaction mass for 2.0 hrs at 25-35° C. Filtered and washed the material with purified water (670 ml) at 25-35° C. Suck dried for 1-2 hrs. Unload the wet material. Dried the material at 55-60° C. for 12 hrs.

Charged Toluene (2200 ml) into the reaction flask and added dried compound of formula (D) under stirring at 20-30° C. Raised the temperature of the reaction mixture upto 105-110° C. and maintained for 1 hr. Cooled the mass to 40-45° C. Filtered and washed the solid material by toluene (12 ml) to obtain Erlotinib. Dry the material at 70-75° C. for 16 hrs.

Yield: Dry wt 89 gm (61%)

HPLC purity: 99.74%

### Step-c) Preparation of Erlotinib Hydrochloride (I)

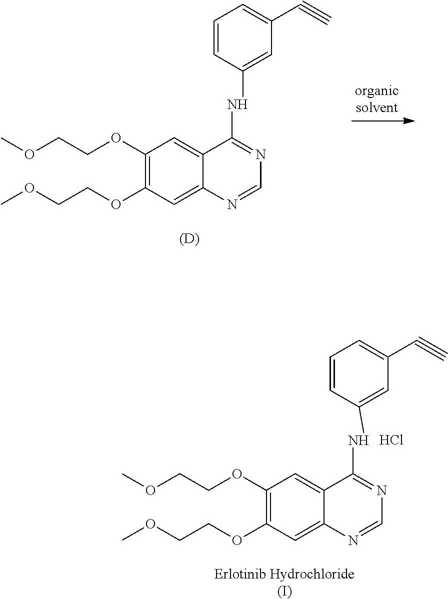

Charged Isopropyl alcohol (2500 ml), Methyl isobutyl ketone (2500 ml) and Erlotinib free base (100 g) into the reaction flask under stirring at 20-30° C. Flushed the reaction flask with Methyl isobutyl ketone (7.2 ml). Raised the temperature of the reaction mixture upto 60-65° C. Stirred the reaction mass for 30 min. Charged activated charcoal (10 gm) and stirred the reaction mass for 30 min at 60-65° C. Filtered the material at 60-65° C. Washed the Hyflo bed with 200 ml 1:1 mixture of IPA (100 ml) and MIB K (100 ml). Filtered the above mother liquor through micron filtration. Transferred and combined main and filtrated mother liquor into the reactor. Maintained the temperature to 60-65° C. Added (already micron filtered) 15% Isopropyl alcohol Hydrochloride solution at 60-65° C. in 2-3 hrs. Stir the reaction mass for 10-15 min. Cool the reaction mass to 25-30° C. Stirred the reaction mass for 2 hrs at 25-30° C. Filtered and washed the material with 200 ml 1:1 mixture of Isopropyl alcohol (100 ml) and Methyl isobutyl ketone (100 ml). Unload the material. Dried the material at 70-75° C. for 20 hrs to obtain Erlotinib hydrochloride.

Yield: Dry wt 100 gm (92%)

HPLC purity: >99.8%

The above mentioned examples, which are provided by way of illustration, should not be construed as limiting the scope of the invention with respect to parameter/s, ingredient/s and quantities use etc.

1. A process of preparing of substantially pure crystalline Erlotinib hydrochloride (I) 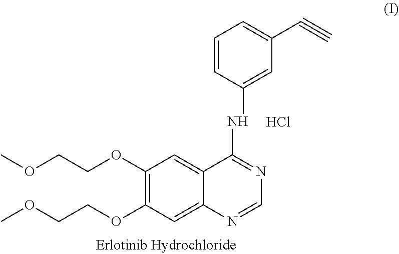 [SMILES: C#Cc1cccc(Nc2ncnc3cc(OCCOC)c(OCCOC)cc23)c1 ; Cl]
comprising the steps of:
a) aminating the compound of formula (A) at temperature ranging between 45-65° C. in the presence of alkali metal dithionite and an organic solvent to obtain the compound of formula (B); 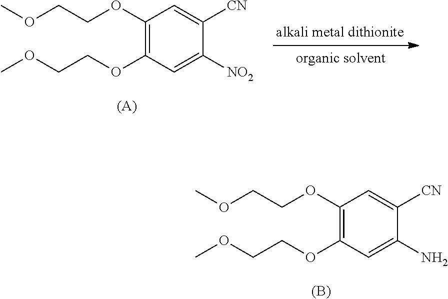
b) reacting compound of formula (B) with N, N-Dimethyl formamide dimethyl acetal (DMFDMA) in the presence of non-polar organic solvent and an organic acid to get an intermediate of compound of formula (C), which in-situ reacted with ethynylaniline hydrochloride to isolate the compound of formula (D) and 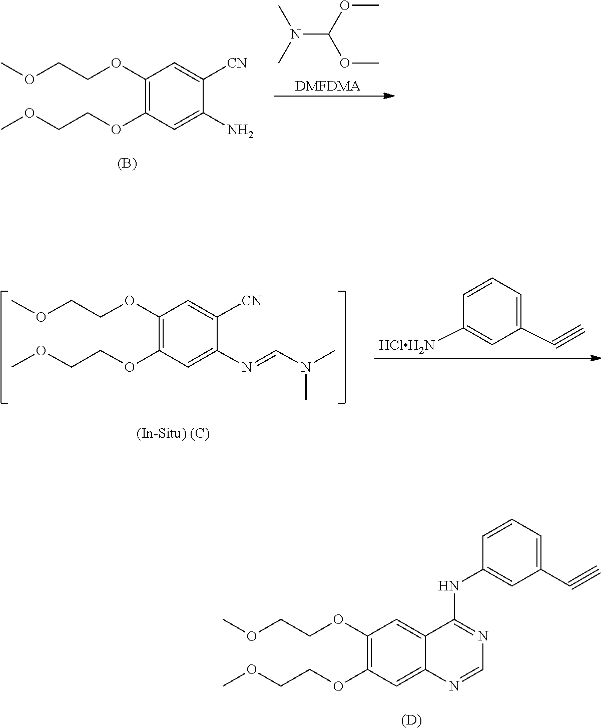
c) hydrochlorinating the compound of formula (D) in a polar organic solvent to get Erlotinib hydrochloride (I).

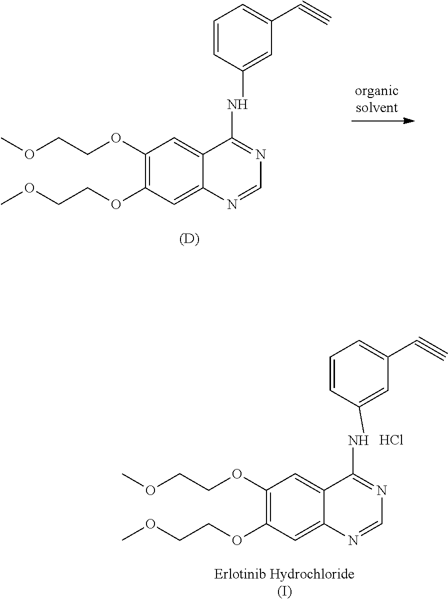

2. A process of preparing of the compound of formula (B) according to claim 1, wherein organic solvent used in step a) is selected from ethylacetate, dichloromethane, chloroform, carbon tetrachloride or a combination thereof.

3. A process of preparing of the compound of formula (B) according to claim 1, in step (a) wherein alkali metal dithionite used is selected from sodium or potassium dithionate.

4. A process of preparing of the compound of formula (C) according to claim 1, wherein non-polar organic solvent in step (b) is selected from toluene, ethylbenzene or xylene.

5. A process of preparing of the compound of formula (D) according to claim 1, wherein organic acid in step (b) is selected from aliphatic acid like acetic acid, propionic acid, butanoic acid, citric acid or tartaric acid.

6. A process of preparing of Erlotinib and its hydrochloride salt of formula (I) according to claim 1, wherein polar organic solvent used in step (c) is selected from isopropanol, ethanol or dimethylformamide.

7. Substantially pure Erlotinib hydrochloride according to claim 1 having purity of greater than 99.8% (% w/w by HPLC).

8. A process of preparing of substantially pure crystalline form of Erlotinib hydrochloride according to claim 1, wherein the crystalline form designated as Form SEA is characterized by XRPD diffraction angle peaks at 5.72, 9.89, 11.39, 18.95, 22.84, 23.60, 24.28, 25.47 and 29.31±0.2 θ°.

9. Thermodynamically stable crystalline Form SEA is characterized by
i). XRPD diffraction angle peaks at 5.72, 9.89, 11.39, 18.95, 22.84, 23.60, 24.28, 25.47 and 29.31±0.2 θ°;
ii). DSC thermogram having thermal peak at ranging between 213-219° C. and
iii). purity greater than 99.8% (% w/w by HPLC).
